(19) United States
(12) Patent Application Publication
(10) Pub. No.: US 2012/0245417 A1
(43) Pub. Date: Sep. 27, 2012

(54) SURGICAL RETRACTOR AND USE THEREOF FOR A THORACOSCOPIC OPERATION

(75) Inventors: Holger Winterberg, Berlin (DE); Marsha Wilke, Berlin (DE); Nils Gelbert, Berlin (DE); Udo Weisse, Heppenheim (DE)

(73) Assignee: W.O.M. WORLD OF MEDICINE AG, Berlin (DE)

(21) Appl. No.: 13/069,444

(22) Filed: Mar. 23, 2011

(30) Foreign Application Priority Data
Mar. 23, 2010 (DE) 10 2010 012 586.5

(51) Int. Cl. A61B 1/32; A61B 1/313

(52) U.S. Cl. 600/114

(57) ABSTRACT

The present patent application relates to a novel surgical retractor and the use thereof for thoracoscopic operations.

The present patent application relates to a novel surgical retractor and the use thereof for thoracoscopic operations.

### BACKGROUND AND PRIOR ART

With increasing frequency, heart and thorax surgeons perform endoscopic operations. For reasons of space, during such operations only one lung is ventilated in the thorax interior. This so-called one-lung ventilation bears risks for the patient and reduces the minimal invasivity of the operational approach. In order to give an example, the respective patient group are in part patients with a heavily constrained heart function, so that the thoracic pressure conditions under one-lumen ventilation significantly impair the hemodynamics, and increase the risk for the patient. In order to minimize this, the instrument according to the invention has been developed. The main function thereof is to hold back the ventilated left-hand lung during an operation in the thorax. This may be important for instance for the cardiac pacemaker technology, the cardiac resynchronization therapy, wherein an epicardiac pacemaker probe is directly fixed on the heart, and by the invention a safe application of the electrode is made possible.

From prior art, so-called endoscopic fan retractors are known, which comprise a fan composed of several lamellae. The different types are finger retractors (3 or 5 fingers) and retractors that tension a curvable chain by means of internal ropes (e.g. original made by Snowden Pencer, see also U.S. Pat. No. 6,309,349).

The fan retractors serve for surgical interventions in the abdomen area, and they serve e.g. to keep the liver away. The prior art fan retractors are however not suited for the interventions described above close to the heart, since they could hurt the lung.

### BRIEF DESCRIPTION OF THE DRAWINGS

FIG. 1 is a perspective view of a surgical retractor according to the present invention.

FIG. 2 is a n exploded view of the handle part and the end cap of such device. In this view slide (A), clamping lever (B) and release (C) are not visible.

FIG. 3 is a detailed view of a spatula unit. The fabric cover is not visible in this view.

FIG. 4 is another view of the spatula unit in expanded condition.

FIG. 5 is another view of the handle part with inserted endoscope (I), the tube (II) through which the endoscope is introduced being visible.

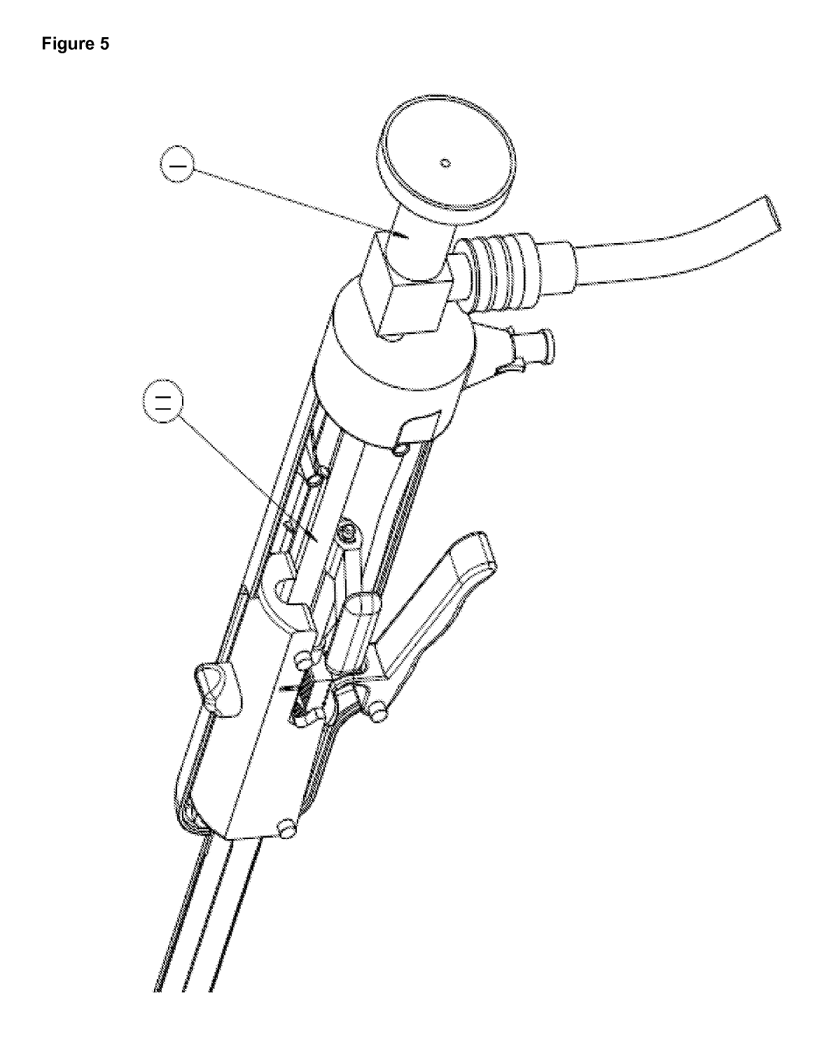

FIG. 6 is a side view of the handle with the release button C.

### DESCRIPTION OF THE INVENTION

It has been found that a surgical retractor comprising a handle at the lateral end and a surface retracting and expanding at the distal end is outstandingly suited to solve the problems explained above.

The surgical retractor according to the invention comprises an elongated shaft (1) and a handle (2). The shaft has an oval shape and contains two continuous lumens. One lumen serves to accommodate a 5-mm endoscope optical system, the other lumen contains an extendible and expandable spatula unit (3). At the proximal end of the shaft, an insufflation tube can be connected. The insufflation gas can flow through both lumens from the proximal end of the shaft to the distal end of the shaft and thus arrives at the area of the operation. The proximal shaft end is firmly connected with the distal handle end.

The handle comprises operating members, by means of which the spatula unit can be extended, bent up and folded back together. Further, the handle comprises all mechanical components that are necessary for carrying-out the operating functions. The end cap of the handle (2.3) is configured such that an inserted endoscope (I) latches and is fixed against translational and rotary movements. In the handle and the end cap, a tube (II) is arranged, through which the endoscope can be introduced without problems into the shaft. The little gap between endoscope and tube simultaneously acts as a seal. The end cap is manufactured in different lengths, in order to allow the use of standard optical systems having different lengths. The end cap is configured such that it can accommodate a standard Luer-Lock connector of the insufflation tube.

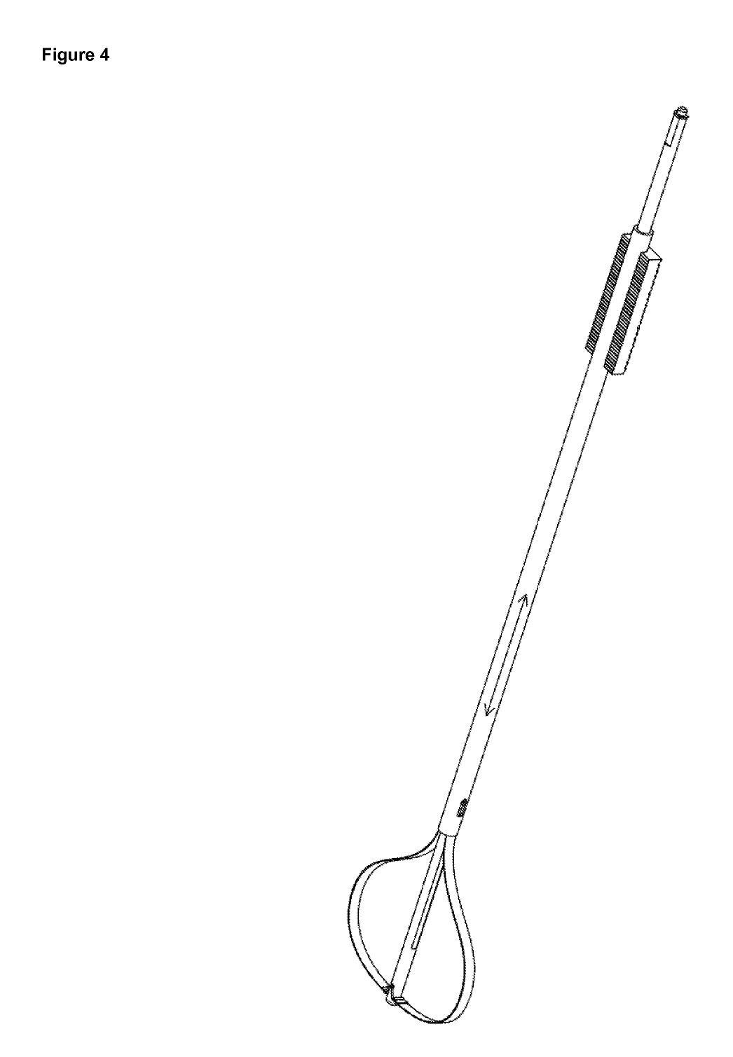

The spatula unit comprises a push rod (3.1) mounted in a tube (3.2). The outer diameter of the push rod is smaller than the inner diameter of the tube. Both parts can move relative to one another. The distal ends of push rod and tube are connected by the two expanding blades (3.4). If the distance of the distal ends of push rod and inner tube is increased, the expanding blades will closely rest against the push rod. The spatula is closed. If by relative movement the distance between the distal ends of push rod and inner tube is reduced, the expanding blades will bulge out. The spatula is open (FIG. 4). A cross-bar fixes the expanding blades at the inner tube and prevents rotation of the parts with respect to one another. A highly elastic fabric is stretched over the expanding blades providing a closed surface of the spatula, causing the retraction of the lung when in use.

The handle serves to operate the surface and allows the accommodation of different commercial 5-mm optical systems.

The actual retractor is formed by the expanded surface of the spatula (3). Depending on the use of the device (see below), the surface may have different sizes, e.g. a maximum diameter of 5 to 20 cm.

### Uses

The retractor according to the invention is preferably used in the field of thoracic surgery. For this purpose, the instrument is introduced in the closed condition through an incision in the chest into the thorax. By means of an operating member A, the closed spatula unit is advanced in a controlled manner. By means of an operating member B, the spatula unit is expanded in a controlled manner, and the expanding blades are locked, so that the spatula unit remains in the respective expanded condition. The advanced and expanded spatula keeps tissue and organs out of the area of operation, so that a free view of the desired region is secured. For this purpose, the spatula must be able to withstand a resistance of approx. 25 mm Hg. By means of an operating member C, the spatula unit can be folded back together, by suspension of the locking, so far that the instrument can be retracted without problems from the thorax. Normally, the spring tension of the expanding blades is sufficient to effect the complete closure of the spatula unit after actuating the operating member C. All steps are carried out under visual inspection by means of an endoscope system introduced into the device.

Further applications of the retractor are possible, for instance for endoscopic operations in the abdomen, wherein for instance a retraction of the liver, the intestine or other organs is required.

Compared to the prior art fan retractors, the retractor according to the invention has a number of advantages. Thereto belongs the lower risk of injury allowing the use close to the heart, it is however also advantageous in other applications. Another advantage is the possibility of a simple handling. For instance, operation by one hand is possible, so that the surgeon in charge has his second hand free for other purposes. In particular notable is the simple possibility (by suspension of the locking mentioned above, whereupon the spatula returns to its original condition by the spring action of the expanding blades) to quickly fold back the expanded retractor by operating the operating member C (release button), so as to allow removal of the instrument. This is in particular important in the case of intraoperative emergency cases.

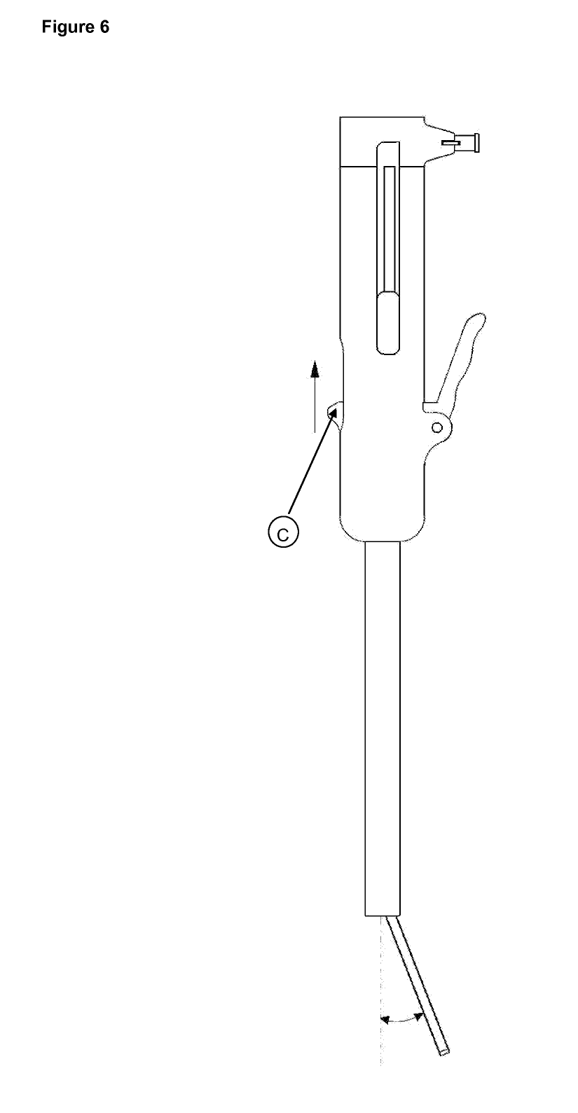

In another embodiment of the invention, the spatula can be rotated in the expanded condition by 30 to 60° out of the axis of the instrument. The deflection is effected by the internal mechanical system. This optional deflection device, too, can be operated in an simple way by one hand and can be reset in particular by actuation of the (one) release button C in the case of an intraoperative emergency situation. A representation of such a potential embodiment is shown in FIG. 6. By actuating the operating member C (release button) in the direction of the arrow, the deflected spatula is folded by spring action back again into the axis of the instrument (broken line), so that the instrument can easily be removed in case of need.

Alternatively, the spatula may also be expanded by fanning-out lamellae. These lamellae, in turn, may be covered by a fabric. With full expansion of the fan, the length of the longest lamella may be 100 mm. Preferably, the length will be 80 mm max. The maximum width of the fan is 150 to 180 mm. The maximum fan angle in relation to the longitudinal axis of the retractor shaft is 45° on either side.

The total diameter of the retractor in the region of the shaft is 8 to 12 mm, preferably 11 mm. The shaft may have a length from 300 to 400 mm.

The retractor according to the invention comprises in the refractor shaft a working channel for receiving a commercially available optical system. This may either be a commercially available rod lens system with 5 mm diameter and 30° recording angle and 350 mm length. Such a rod lens system allows the visual evaluation of the area of operation. Alternatively, a flexible endoscope or a rigid or flexible endoscope with a video picture sensor (e.g. CCD or C-MOS) at the distal end may also be used.

In another improvement of the retractor according to the invention, the shaft contains another working channel that can be used for insufflation. The optional insufflation channel serves, for instance, to keep the ventilated lung away from the area of operation. Alternatively or additionally, the retractor may optionally comprise another working channel, for instance for the introduction of a light conductor for illuminating the area of operation.

### Manufacture

Manufacture of the retractor according to the invention is achieved in a conventional way by using biocompatible materials. The manufacture of the solid plastic parts is usually made by injection molding. For the metal parts preferably stainless steel is used. The fabric cover is preferably made of a nylon-elastane blend (e.g. 80% polyamide (nylon), 20% (block copolymer of polyurethane and polyethylene glycol (elastane)). Of course, all parts of the retractor must be capable of being sterilized.

The embodiment of a retractor according to the invention shown exemplarily in the drawings is composed of the following parts:

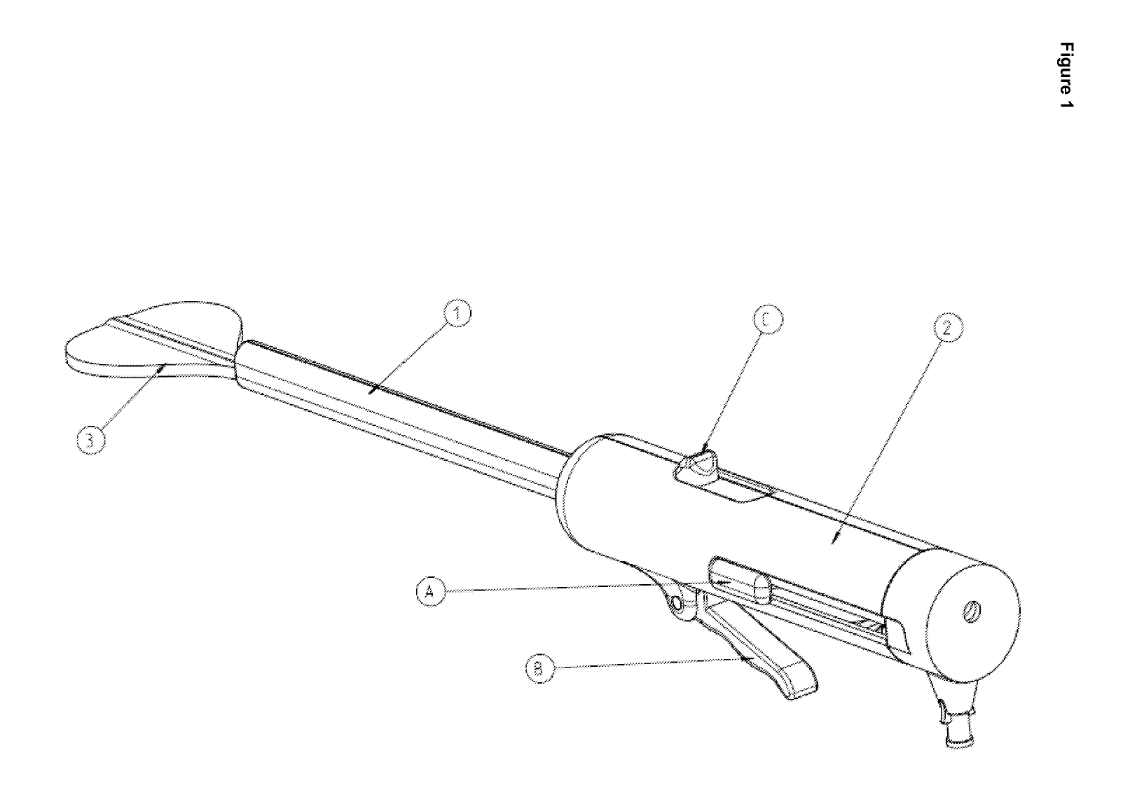

(FIG. 1)

| | |
|---|---|
| instrument shaft | (1) |
| handle | (2) |
| spatula unit | (3) |
| purchased parts | (4) |

The instrument shaft (1) is composed of the following parts:

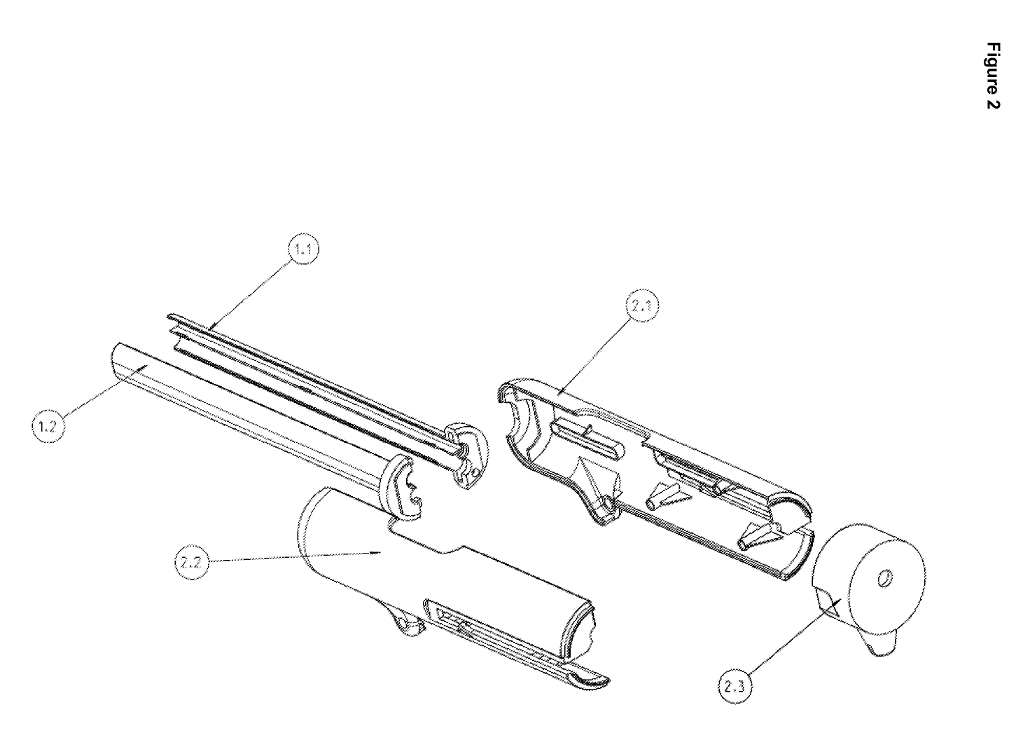

(FIG. 2)

| | |
|---|---|
| right-hand shaft half | (1.1) |
| left-hand shaft half | (1.2) |

The handle (2) is composed of the following parts:

(FIG. 2)

| | |
|---|---|
| right-hand handle half | (2.1) |
| left-hand handle half | (2.2) |
| end cap | (2.3) |

At the handle (2) there are arranged the following operating members:

(FIG. 1)

| | |
|---|---|
| slide | (A) |
| clamping lever | (B) |
| release | (C) |

The spatula unit (3) is composed of the following parts:

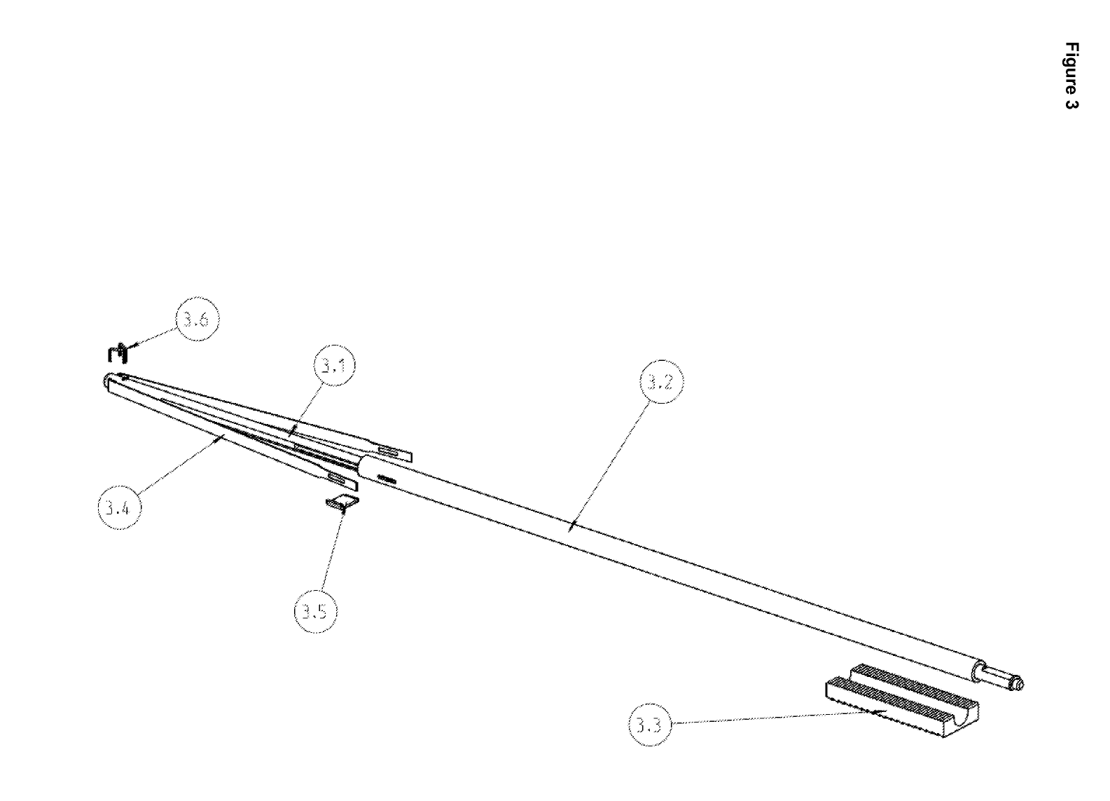

(FIG. 3)

| | |
|---|---|
| push rod | (3.1) |
| inner tube | (3.2) |
| tooth profile | (3.3) |
| expanding blade | (3.4) |
| crossbar | (3.5) |
| spatula bracket | (3.6) |
| fabric cover | (3.7) |

The instrument comprises the following purchased parts:

| | |
|---|---|
| PVC tube (ID × OD) 2 × 3.4 mm | (4.1) |
| Luer-Lock 71350 | (4.2) |
| retaining washer DIN 6799-1,9 | (4.3) |
| stainless steel tube 5.5 × 0.2 | (4.4) |
| material 1.4301 | |

Alternative embodiments are imaginable and can be designed without inventive activity.

1. A surgical retractor comprising a shaft and a handle, an expandable spatula located at the distal end of the handle and at least one further working channel for receiving an optical system, the expandable spatula being mounted at the distal end of a push rod that is arranged in a tube, the push rod being movable relative to the handle, the spatula being expanded by expanding blades in the case of a reduction of the distance between tube and push rod,
wherein a highly elastic fabric is stretched over the spatula, said fabric having in the expanded condition a closed surface, by means of which a transfer of force onto inner organs is made possible.

2. The surgical retractor according to claim 1, wherein the expanding blades are locked in the expanded condition.

3. The surgical retractor according to claim 2, wherein the locking of the expanding blades is suspended in a controlled manner by operating the operating member C, whereupon the expanding blades are spontaneously reset by their spring action into the original condition, so that the retractor can be removed.

4. The surgical retractor according to claim 1, wherein the highly elastic fabric covering the spatula consists of polyamide, a block copolymer of polyurethane and polyethylene glycol or a blend thereof.

5. The surgical retractor according to claim 1, wherein at least one further working channel.

6. The use of a surgical retractor according to claim 1 in thoracoscopy.

7. The use of a surgical retractor according to claim 1, for the implantation of a cardiac pacemaker.

8. The use of a surgical retractor according to claim 1 in laparoscopy and pelviscopy.

9. The use of a surgical retractor according to claim 2 in thoracoscopy.

10. The use of a surgical retractor according to claim 2 for the implantation of a cardiac pacemaker.

11. The use of a surgical retractor according to claim 2 in laparoscopy and pelviscopy.

12. The use of a surgical retractor according to claim 3 in thoracoscopy.

13. The use of a surgical retractor according to claim 3 for the implantation of a cardiac pacemaker.

14. The use of a surgical retractor according to claim 3 in laparoscopy and pelviscopy.

15. The use of a surgical retractor according to claim 4 in thoracoscopy.

16. The use of a surgical retractor according to claim 4 for the implantation of a cardiac pacemaker.

17. The use of a surgical retractor according to claim 4 in laparoscopy and pelviscopy.

18. The use of a surgical retractor according to claim 5 in thoracoscopy.

19. The use of a surgical retractor according to claim 5 for the implantation of a cardiac pacemaker.

20. The use of a surgical retractor according to claim 5 in laparoscopy and pelviscopy.
